(19) United States
(12) Patent Application Publication
(10) Pub. No.: US 2025/0249178 A1
(43) Pub. Date: Aug. 7, 2025

(54) Automated Syringe

(71) Applicant: Becton, Dickinson and Company, Franklin Lakes, NJ (US)

(72) Inventors: Mukilan Balamurugan, Theni (IN); Edwin Jeyaseelan Arockia Doss, Madurai (IN)

(21) Appl. No.: 18/855,709

(22) PCT Filed: Apr. 10, 2023
(86) PCT No.: PCT/US2023/018014

§ 371 Date: Oct. 10, 2024

Related U.S. Application Data
(60) Provisional application No. 63/329,641, filed on Apr. 11, 2022.

(51) Int. Cl. A61M 5/315; A61M 5/20; A61M 5/31; A61M 5/50

(52) U.S. Cl. CPC A61M 5/31501; A61M 5/20; A61M 5/31566; A61M 5/31591; A61M 5/50; A61M 2005/2073; A61M 2005/3143; A61M 2005/5073; A61M 2205/273; A61M 2205/8281

(57) ABSTRACT

A syringe comprising a syringe barrel including an open distal end, a proximal end, and a sidewall extending therebetween and defining a chamber; a stopper disposed within the chamber in sealing engagement with the sidewall; a medicinal fluid filling the chamber of the syringe barrel between the stopper and the open distal end of the chamber; a biasing member configured to move the stopper from a first position to a second position; a locking assembly comprising a proximal end portion covering the proximal end of the syringe and a locking member connected to the stopper. At least a portion of the locking member engages the proximal end portion to hold the stopper in the first position, disengagement of the portion of the locking member from the proximal end portion frees the stopper, and the stopper is moved to the second position by the biasing member.

### CROSS-REFERENCE TO RELATED APPLICATION

The present application claims priority to U.S. Provisional Application Ser. No. 63/329,641, entitled “Automated Syringe”, filed Apr. 11, 2022, the entire disclosure of which is hereby incorporated by reference in its' entirety.

### BACKGROUND OF THE INVENTION

### Field of the Invention

The present invention is directed to a syringe that is configured to be filled with a medicinal fluid and, more specifically, to a syringe that automatically expels a medicinal fluid from the syringe without the use of a manual plunger rod and is incapable of being reused.

### Description of Related Art

Intravenous catheters are flushed with saline or anticoagulant solutions on a regular basis in order clean out the tube of the catheter and prevent blockage after a patient has received an intravenous infusion through the catheter. Prior art syringes filled with saline or anticoagulant solution are used for such flushing. In such prior art syringes, as shown in FIG. 1, the medicinal fluid is contained within a chamber lof the syringe barrel 2 and forced from the distal open end covered by a cap 3 of the syringe barrel 2 by a stopper 4 that is transitioned from a proximal position in the chamber 1 to a more distal position in the chamber 1 thereby expelling the medicinal fluid from the chamber 1. The stopper 4 is transitioned from the proximal position to the more distal position when the user exerts a distal force on a plunger rod 5 attached to the stopper.

With the prior art syringe, it is therefore possible to reuse the syringe by refilling it with more medicinal fluid and to use the same syringe to flush the intravenous catheters of more than one patient by flushing the catheter of a first patient using a portion of the medicinal fluid and then flushing the catheter of a second patient using the remainder of the medicinal fluid. It has been found that, in cases where the same syringe is used to flush the catheters of different patients, diseases, such as hepatitis C, can be transferred from the first patient to the second patient.

In addition, the packaging footprint of the prior art syringes is large because the plunger rod 5 must extend out of the syringe barrel 2 in order to provide room for the chamber 1 to be filled with the medicinal fluid.

Further, the prior art syringes require high manual force to expel the medicinal fluid into the intravenous catheter at the time of flushing.

Against this background, it is the object of the current invention to provide a syringe that is restricted to a single use, has a reduced package size while having the same functionality as prior art syringes, and has similar or reduced injection force to the prior art syringes.

### SUMMARY OF THE INVENTION

The present invention is directed to a syringe comprising a syringe barrel comprising an open distal end, a proximal end, and a sidewall extending between the open distal end and the proximal end and defining a chamber; a stopper disposed within the chamber in sealing engagement with the sidewall; a medicinal fluid filling the chamber of the syringe barrel between the stopper and the open distal end of the chamber; a biasing member configured to move the stopper from a first position to a second position; a locking assembly comprising a proximal end portion covering the proximal end of the syringe and a locking member connected to the stopper. At least a portion of the locking member engages the proximal end portion to hold the stopper in the first position, disengagement of the portion of the locking member from the proximal end portion frees the stopper, and the stopper is moved to the second position by the biasing member.

The locking member may comprise a tab and a connection rod extending between the tab and the stopper. When the locking member is engaged with the proximal end portion, the connection rod extends through an opening in the proximal end portion and the tab engages a proximal exterior surface of the proximal end portion.

In a first embodiment of the locking member, the tab may be sized and/or shaped such that it cannot pass through the opening. The tab may be releasably attached to the connection rod and detachment of the tab from the connection rod may disengage the locking member from the proximal end portion. The tab may be attached to the connection rod by a breakable connection by which the tab can be detached from the connection rod, and the breakable connection may be a portion between the connection rod and the tab having a cross-sectional area that is less than a cross-sectional area of the connection rod.

When the stopper is in the second position, the connection rod is completely contained within the chamber.

In a second embodiment of the locking member, at least a portion of the tab has a shape corresponding to a shape of at least a portion of the opening, and the locking member is transitionable from a locked position, where the tab of the locking member engages the proximal end portion to a release position where the tab of the locking member passes through the opening in the proximal end portion. In the locked position, the biasing member provides a downward force on the tab to hold it in place against the proximal end portion. Rotation of the locking member with respect to the proximal end portion transitions the locking member from the locked position to the released position. The portion of the opening may be a slit and the portion of the tab may be a disk. In the locked position, the tab may be perpendicular to the slit, and in the released position, the tab may be parallel to the slit.

When the stopper is in the second position, the locking member is completely contained within the chamber.

In a third embodiment of the locking member, the proximal end portion is contained within the chamber at the proximal end of the syringe barrel and the locking member further comprises a locking member cap covering the proximal end portion, the locking member cap engaging the tab. A portion of the locking member cap may extend about an outer perimeter of the syringe barrel. The portion of the locking member cap extending about the outer perimeter of the syringe barrel may include ridges to aid a user in gripping the locking member cap. Rotation of the locking member cap with respect to the syringe barrel transitions the locking member from the locked position to the release position. Protrusions may extend from the locking member cap to engage the tab. The protrusions may define a recess having a cross-section corresponding to a cross-section of the tab, and the cross-sections of the recess and the tab may be cross-shaped. The portion of the opening may be a slit and the portion of the tab may be a bar, and the bar may be perpendicular to the connection rod such that the bar and the connection rod form a T-shape.

When the stopper is in the second position, the tab and the connection rod are completely contained within the chamber.

With any of the above described embodiments, the connection rod may have a cross-shaped cross-section. The proximal end portion may comprise a distal portion that is received within the chamber of the syringe barrel. With the distal portion of the proximal end portion received within the syringe barrel, a proximal exterior surface of the proximal end portion is flush with the proximal end of the syringe barrel.

A frictional force provided between the stopper and the sidewall of the syringe barrel is set to a predetermined value and a biasing force exerted by the biasing member on the stopper is set to a predetermined value to control a rate of movement of the stopper from the first position to the second position at a predetermined value. The biasing member may be configured to apply a biasing force to the stopper that is greater than an opposing force exerted on the stopper, where the opposing force is a sum of the frictional force between the stopper and the sidewall of the syringe barrel, the venous pressure coming from the attachment of the syringe to a vein of a patient, and the fluid pressure of the medicinal fluid contained in the chamber of the syringe barrel and/or the biasing member may be configured to provide a predetermined biasing force that in combination with an inner diameter of the syringe barrel causes the medicinal fluid to be expelled from the open distal end of the syringe body at a predetermined force.

The medicinal fluid may be saline or an anti-coagulant solution.

The stopper moves in a continuous manner from the first position to the second position expelling the fluid from the syringe in a continuous flow.

The biasing member may comprise a spring and/or a viscous damper.

The syringe may further comprise a removable tip cap covering the open distal end of the syringe barrel, and/or the open distal end of the syringe barrel is configured for attachment to an intravenous catheter and may be a luer lock tip.

Further embodiments or aspects are set forth in the following numbered clauses:

Clause 1: A syringe comprising: a syringe barrel comprising an open distal end, a proximal end, and a sidewall extending between the open distal end and the proximal end and defining a chamber; a stopper disposed within the chamber in sealing engagement with the sidewall; wherein the syringe is configured to hold a medicinal fluid in the chamber of the syringe barrel between the stopper and the open distal end of the chamber, characterized in that the syringe further comprises a biasing member configured to move the stopper from a first position to a second position; and a locking assembly comprising a proximal end portion covering the proximal end of the syringe and a locking member connected to the stopper, wherein at least a portion of the locking member engages the proximal end portion to hold the stopper in the first position, disengagement of the portion of the locking member from the proximal end portion frees the stopper, and the stopper is moved to the second position by the biasing member.

Clause 2: The syringe of clause 1, wherein the locking member comprises a tab and a connection rod extending between the tab and the stopper.

Clause 3: The syringe of clause 2, wherein, when the locking member is engaged with the proximal end portion, the connection rod extends through an opening in the proximal end portion and the tab engages a proximal exterior surface of the proximal end portion.

Clause 4: The syringe of clause 3, wherein the tab is sized and/or shaped such that the tab cannot pass through the opening.

Clause 5: The syringe of any of clauses 2-4, wherein the tab is releasably attached to the connection rod and detachment of the tab from the connection rod disengages the locking member from the proximal end portion.

Clause 6: The syringe of clause 5, wherein the tab is attached to the connection rod by a breakable connection by which the tab can be detached from the connection rod.

Clause 7: The syringe of clause 6, wherein the breakable connection is a portion between the connection rod and the tab having a cross-sectional area that is less than a cross-sectional area of the connection rod.

Clause 8: The syringe of any of clauses 2-7, wherein, with the stopper in the second position, the connection rod is completely contained within the chamber.

Clause 9: The syringe of clause 3, wherein at least a portion of the tab has a shape corresponding to a shape of at least a portion of the opening, and the locking member is transitionable from a locked position, where the tab of the locking member engages the proximal end portion to a release position where the tab of the locking member passes through the opening in the proximal end portion.

Clause 10: The syringe of clause 9, wherein rotation of the locking member with respect to the proximal end portion transitions the locking member from the locked position to the released position.

Clause 11: The syringe of clause 9 or clause 10, wherein, with the stopper in the second position, the tab and the connection rod are completely contained within the chamber.

Clause 12: The syringe any of clauses 9-11, wherein the portion of the opening is a slit and the portion of the tab is a disk.

Clause 13: The syringe of clause 9, wherein the proximal end portion is contained within the chamber at the proximal end of the syringe barrel and the locking member further comprises a locking member proximal end portion covering the proximal end portion, the locking member proximal end portion engaging the tab.

Clause 14: The syringe of clause 13, wherein a portion of the locking member cap extends about an outer perimeter of the syringe barrel.

Clause 15: The syringe of clause 14, wherein the portion of the locking member cap extending about the outer perimeter of the syringe barrel includes ridges to aid a user in gripping the locking member cap.

Clause 16: The syringe of any of clauses 13-15, wherein rotation of the locking member cap with respect to the syringe barrel transitions the locking member from the locked position to the release position.

Clause 17: The syringe of any of clauses 13-16, wherein protrusions extend from the locking member cap and engage the tab.

Clause 18: The syringe of clause 17, wherein the protrusions define a recess having a cross-section corresponding to a cross-section of the tab.

Clause 19: The syringe of clause 18, wherein the cross-sections of the recess and the tab are cross-shaped.

Clause 20: The syringe of any of clauses 13-19, wherein the portion of the opening is a slit and the portion of the tab is a bar.

Clause 21: The syringe of clause 20, wherein the bar is perpendicular to the connection rod such that the bar and the connection rod form a T-shape.

Clause 22: The syringe of any of clauses 13-21, wherein, with the stopper in the second position, the tab and the connection rod are completely contained within the chamber.

Clause 23: The syringe of any of clauses 2-22, wherein the connection rod has a cross-shaped cross-section.

Clause 24: The syringe of any of clauses 1-23, wherein the proximal end portion comprises a distal portion that is received within the chamber of the syringe barrel.

Clause 25: The syringe of clause 24, wherein, with the distal portion of the proximal end portion received within the syringe barrel, a proximal exterior surface of the proximal end portion is flush with the proximal end of the syringe barrel.

Clause 26: The syringe of any of clauses 1-25, wherein a frictional force provided between the stopper and the sidewall of the syringe barrel is set to a predetermined value and a biasing force exerted by the biasing member on the stopper is set to a predetermined value to control a rate of movement of the stopper from the first position to the second position at a predetermined value.

Clause 27: The syringe of any of clauses 1-26, wherein the biasing member is configured to apply a biasing force to the stopper that is greater than an opposing force exerted on the stopper, where the opposing force is a sum of the frictional force between the stopper and the sidewall of the syringe barrel, the venous pressure coming from the attachment of the syringe to a vein of a patient, and the fluid pressure of the medicinal fluid contained in the chamber of the syringe barrel.

Clause 28: The syringe of any of clauses 1-27, wherein the biasing member is configured to provide a predetermined biasing force that in combination with an inner diameter of the syringe barrel causes the medicinal fluid to be expelled from the open distal end of the syringe body at a predetermined force.

Clause 29: The syringe of any of clauses 1-28, wherein the medicinal fluid is saline or an anti-coagulant solution.

Clause 30: The syringe of any of clauses 1-29, wherein the stopper moves in a continuous manner from the first position to the second position expelling the fluid from the syringe in a continuous flow.

Clause 31: The syringe of any of clauses 1-30, wherein the biasing member comprises a spring.

Clause 32: The syringe of any of clauses 1-31, wherein the biasing member comprises a viscous damper.

Clause 33: The syringe of any of clauses 1-32, further comprising a removable tip cap covering the open distal end of the syringe barrel.

Clause 34: The syringe of any of clauses 1-33, wherein the open distal end of the syringe barrel is configured for attachment to an intravenous catheter.

Clause 35: The syringe of any of clauses 1-34, wherein the open distal end of the syringe barrel comprises a luer lock tip.

Clause 36: The syringe of any of clauses 1-35, wherein the syringe is pre-filled with the medicinal fluid.

### BRIEF DESCRIPTION OF THE DRAWINGS

FIG. 1 is a side view of a prior art syringe;

FIG. 2 is a side view of a first embodiment of a syringe according to the invention where the syringe has not yet been filled with a medicinal fluid and the stopper is in the first (pre-use) position;

FIG. 3 is an opposite side view of the syringe of FIG. 1, where the syringe contains a medicinal fluid and the stopper is in the first (pre-use) position;

FIG. 4 is an opposite side view of the syringe of FIG. 1, where the medicinal fluid has been expelled from the syringe and the stopper is in the second (post-use) position;

FIG. 5 is a top view of the syringe of FIG. 1 prior to use;

FIG. 6 is a top view of the syringe of FIG. 1 after use;

FIG. 7 is a side view of the proximal end portion, biasing member, locking member, and stopper syringe of FIG. 1 prior to use;

FIG. 8 is a side view of the proximal end portion, biasing member, locking member, and stopper of the syringe of FIG. 1 after use;

FIG. 9 is a side view of the locking member of the syringe of FIG. 1;

FIG. 10 is a side perspective view of a second embodiment of a syringe according to the invention where the syringe has not yet been filled with a medicinal fluid and the stopper is in the first (pre-use) position;

FIG. 11 is a side perspective view of the syringe of FIG. 10, where the medicinal fluid has be expelled from the syringe and the stopper is in the second (post-use) position;

FIG. 12 is a top view of the syringe of FIG. 10 prior to use;

FIG. 13 is a top view of the proximal end portion of the syringe of FIG. 10;

FIG. 14 is a side perspective view of the proximal end portion of the syringe of FIG. 10;

FIG. 15 is a side view of the connection rod of the syringe of FIG. 10;

FIG. 16 is a side perspective view of a third embodiment of a syringe according to the invention where the syringe has not yet been filled with a medicinal fluid and the stopper is in the first (pre-use) position;

FIG. 17 is a side perspective view of the syringe of FIG. 16, where the medicinal fluid has been expelled from the syringe and the stopper is in the second (post-use) position;

FIG. 18 is a top view of the syringe of FIG. 16;

FIG. 19 is a bottom view of the locking member cap of the syringe of FIG. 16 showing the engagement of the locking member cap with the tab of the locking member prior to use;

FIG. 20 is a top view of the proximal end portion of the syringe of FIG. 16 prior to use; and

FIG. 21 is a side view of the locking member of the syringe of FIG. 16.

### DESCRIPTION OF THE INVENTION

As used herein, “including”, “such as”, “for example” and like terms mean “including/such as/for example but not limited to”.

The present invention is directed to a syringe that is configured to hold a medicinal fluid and expel the medicinal fluid automatically without the use of a manually actuated plunger rod and is incapable of being reused. The syringe may be pre-filled with the medicinal fluid. The syringe is particularly useful for injecting saline or an anticoagulant solution into an intravenous catheter to flush the catheter, thereby clearing and/or preventing blockage of the catheter.

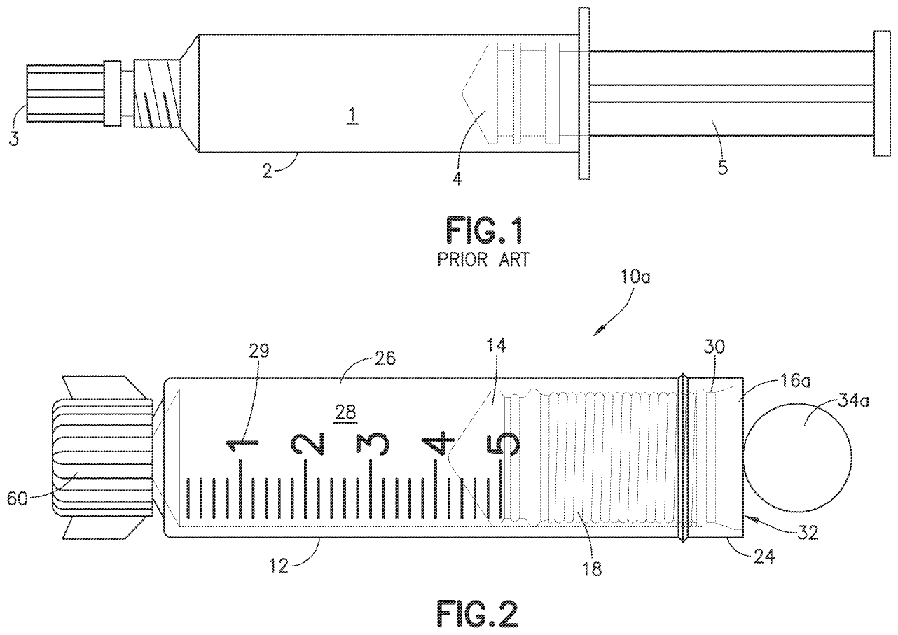

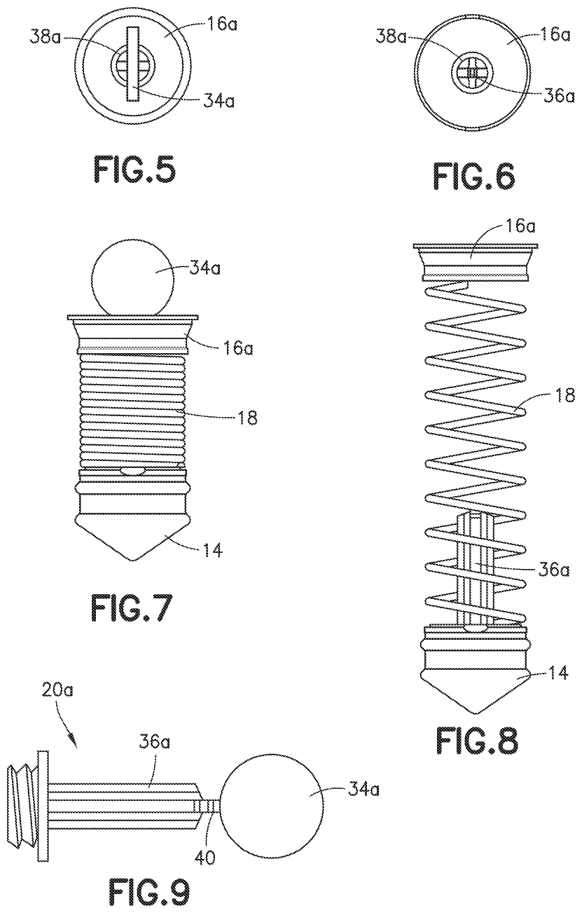

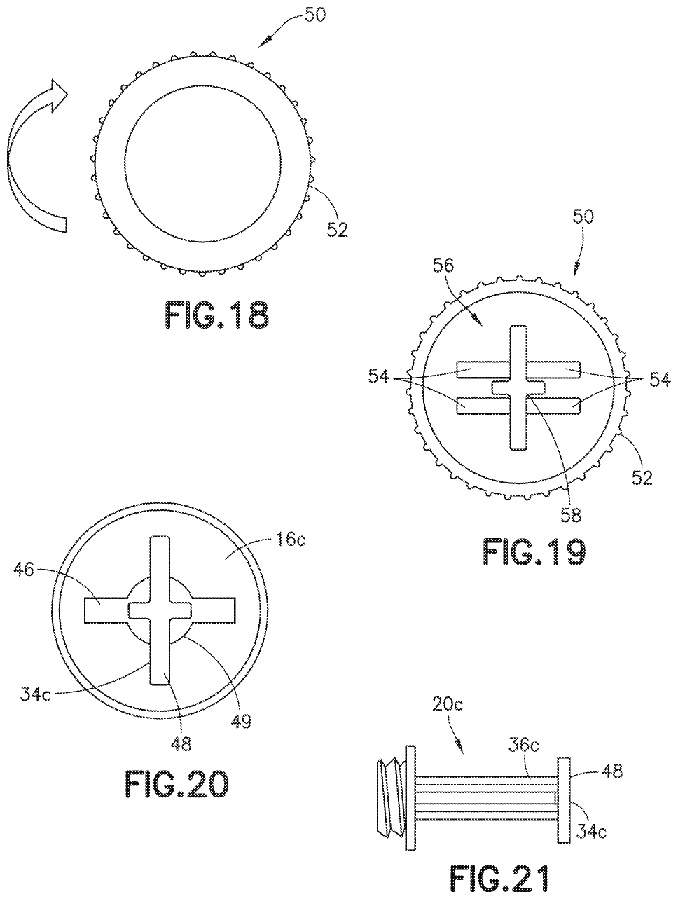

As shown in FIGS. 1-21, the syringe 10a, 10b, 10c comprises a syringe barrel 12, a stopper 14, a biasing member 18, and a locking assembly comprising a proximal end portion 16a, 16b, 16c and a locking member 20a, 20b, 20c. The syringe is configured to be filled with a medicinal fluid 21.

The syringe barrel 12 comprises an open distal end 22, a proximal end 24, and a sidewall 26 extending between the distal end 22 and the proximal end 24 and defining a chamber 28. The syringe barrel 12 may be transparent to allow the contents of the chamber 28 to be viewed by the user and may include indicia 29 on the outer surface indicating the amount of medicinal fluid 21 contained within the chamber 28. The open distal end 22 of the syringe barrel 12 is configured for attachment to an intravenous catheter and may be a luer lock tip.

The stopper 14 is disposed within the chamber 28 of the syringe barrel 12. The stopper 14 is in sealing engagement with the sidewall 26 of the syringe barrel 12. The stopper 14 may be made of any suitable material including, but not limited to, rubber and silicone.

A proximal end portion 16a, 16b, 16c of the locking assembly covers the proximal end 24 of the syringe barrel 12. The proximal end portion 16a, 16b, 16c includes a portion 30 that extends into the chamber 28 of the syringe barrel 12, and a proximal exterior surface 32 of the proximal end portion 16a, 16b, 16c may be flush with the proximal end 24 of the syringe barrel 12.

The biasing member 18 is configured to move the stopper 14 from a first (pre-use) position to a second (post-use) position. The biasing member 18 may be in any suitable form that is capable of storing energy and releasing the energy to provide a force on the stopper 14 to move the stopper 14 within the syringe barrel 12. For example, the biasing member 18 may be a coil, a compression spring, or a polymer disc spring. The biasing member 18 may also comprise a viscous damper. For example, the viscous damper may be a tacky substance or fluid or a viscous fluid or gel applied to or around the biasing member 18 to slow the initial release and/or the rate of movement of the biasing member 18 by dampening the mass system of the biasing member 18. The biasing member 18 may be located in the chamber 28 of the syringe barrel 12 between the stopper 14 and the proximal end portion 16a, 16b, 16c.

In the first position, the stopper 14 is positioned near the proximal end 24 of the syringe barrel 12, thereby providing a space in the chamber 28 of the syringe barrel 12 in which the medicinal fluid 21 is stored. The biasing member 18 is a state of stored energy. For example, if the biasing member 18 is a spring, the spring is at least partially compressed, for example, between the stopper 14 and the proximal end portion 16a, 16b, 16c.

The transition of the stopper 14 from the first position to the second position occurs when the stored energy of the biasing member 18 is applied to the stopper 14 moving the stopper 14 in a distal direction within the chamber 28 of the syringe barrel 12. For example, if the biasing member 18 is a spring, the spring expands forcing the stopper 14 in a distal direction within the chamber 28 of the syringe barrel 12. Movement of the stopper 14 in the distal direction from the first position to the second position expels the medicinal fluid 21 contained in the chamber 28 of the syringe barrel 12 from the open distal end 22 of the syringe barrel 12.

In the second position, the stopper is positioned at a distal end of the chamber 28 of the syringe barrel 12 and may abut a bottom surface of the chamber 28 of the syringe barrel 12.

The locking member 20a, 20b, 20c is connected to the stopper 14 and engages the proximal end portion 16a, 16b, 16c. Engagement between the locking member 20a, 20b, 20c and the proximal end portion 16a, 16b, 16c holds the stopper 14 in the first position such that the force applied to the stopper 14 by the biasing member 18 cannot move the stopper 14. Disengagement of the locking member 20a, 20b, 20c from the proximal end portion 16a, 16b, 16c releases the stopper 14 allowing the force applied to the stopper 14 by the biasing member 18 to move the stopper 14 from the first position to the second position, expelling the medicinal fluid 21 from the chamber 28 of the syringe barrel 12.

The locking member 20a, 20b, 20c may comprise a tab 34 and a connection rod 36 extending between the tab 34 and the stopper 14. The connection rod 36 may be attached to the stopper 14 in any suitable manner including, but not limited to, a screw connection (FIG. 9), gluing, adhesive bonding, co-injection molding, and the like. The connection rod 36 may have any suitable cross-sectional shape including square, rectangular, triangular, circular, oval, polygonal, and cross-shaped. The length of the connection rod 36 is substantially less than the length of the chamber 28 of the syringe barrel 12, for example, less than half the length of the chamber 28 of the syringe barrel 12, less than one third of the length of the chamber 28 of the syringe barrel 12, or less than one quarter the length of the chamber 28 of the syringe barrel 12.

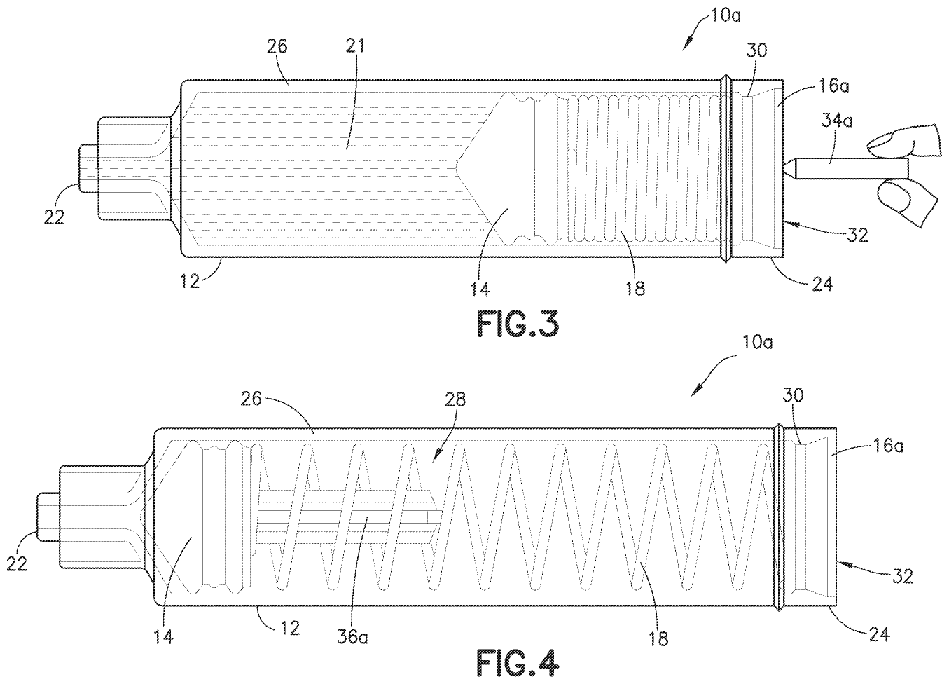

In a syringe having a first embodiment of the locking member 20a, shown in FIGS. 2-9, when the stopper 14 is in the first position (FIGS. 2, 3, 5, and 7), the connection rod 34a extends through an opening 38a in the proximal end portion 16a and the tab 34a engages the exterior top surface 32a of the proximal end portion 16a. With the tab 34a engaging the proximal exterior surface 32a of the proximal end portion 16a, the stopper 14 is held in the first position and cannot move within the chamber 28 of the syringe barrel 12 (FIGS. 2 and 3).

The tab 34a is sized and/or shaped such that it cannot pass through the opening 38a. The opening 38a is a through opening. For example, the opening 38a may be circular and the tab 34a may be a circular disk having a diameter greater than the diameter of the opening 38a.

The tab 34a may also be shaped to provide gripping surfaces for a user to grasp the tab 34a, for example, the large side surfaces of the disk shape of the tab 34a.

The tab 34a is releasably attached to the connection rod 36a and detachment of the tab 34a from the connection rod 36a disengages the locking member 20a from the proximal end portion 16a. The tab 34a may be attached to the connection rod 36a by a breakable connection 40 by which the tab 34a can be detached from the connection rod 36a. For example, the breakable connection 40 may be a portion between the tab 34a and the connection rod 36a having a cross-sectional area that is less than a cross-sectional area of the connection rod 36a such that a bending force applied to the breakable connection 40 by the tab 34a breaks the breakable connection 40 between the tab 34a and the connection rod 36a and separates the tab 34a from the connection rod 36a. When the tab 34a is separated from the connection rod 36a, thereby releasing the engagement of the locking member 20a with the proximal end portion 16a, the stopper 14 is free to move within the chamber 28 of the syringe barrel 12 and the stored energy of the biasing member 18 is released and applied to the stopper 14 moving the stopper 14 in a distal direction within the chamber 28 and expelling the medicinal fluid 21 from the chamber 28 through the open distal end 22 of the syringe barrel 12.

When the stopper 14 is in the second position (FIG. 4) and the medicinal fluid 21 has been expelled from the chamber 28 of the syringe barrel 12, the connection rod 34a of the locking member 20a is completely contained within the chamber 28 of the syringe barrel 12 between the stopper 14 and the proximal end portion 16a.

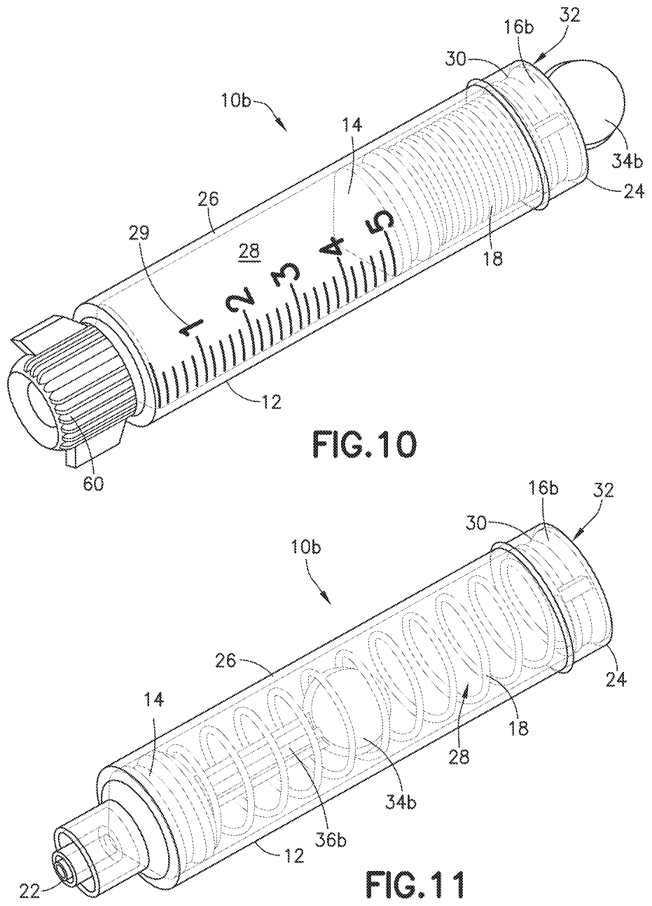

In a syringe having a second embodiment of the locking member 20b, shown in FIGS. 10-15, as in the first embodiment of the locking member 20a, when the stopper 14 is in the first position, the connection rod 34b extends through an opening 38b in the proximal end portion 16b and the tab 34b engages the proximal exterior surface 32b of the proximal end portion 16b. With the tab 34b engaging the proximal exterior surface 32a of the proximal end portion 16b, the stopper 14 is held in the first position and cannot move within the chamber 28 of the syringe barrel 12.

At least a portion of the tab 34b has a shape corresponding to the shape of at least a portion of the opening 38b, and the locking member 20b is transitionable from a locked position, where the tab 34b of the locking member 20b engages the proximal exterior surface 32b of the proximal end portion 16b, to a release position, where the tab 34b of the locking member 20b passes through the opening 38b in the proximal end portion 16b. The portion of the opening 38b may be a slit 42 and the portion of the tab 34b may be a corresponding disk. The opening 38b may further include a circular portion 44 through which the connection rod 36b extends.

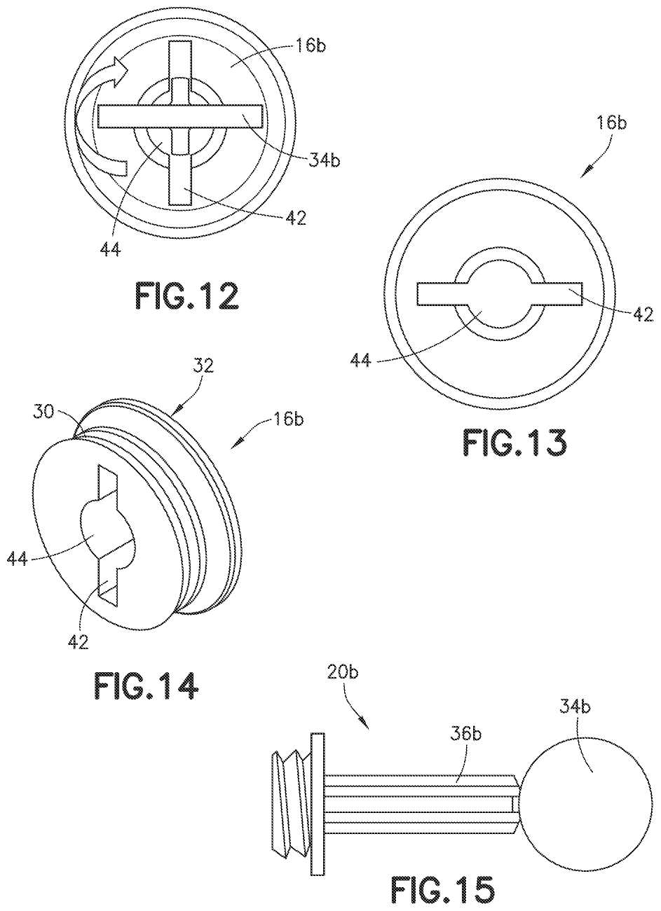

With the tab 34b positioned perpendicular to the slit 42 (FIG. 12), the tab 34b engages the proximal exterior surface 32b of the proximal end portion 16b, the stopper 14 is held in the first position and cannot move within the chamber 28 of the syringe barrel 12 (FIG. 10). With the tab 34b positioned parallel to the slit 42, the tab 34b can pass through the opening 38b and into the chamber 28 of the syringe barrel 12 (FIG. 11). By rotating the locking member 20b with respect to the proximal end portion 16b (FIG. 12), the locking member 20b can be transitioned from a locked position, where the tab 34b of the locking member 20b engages the proximal exterior surface 32b of the proximal end portion 16b, to a release position, where the tab 34b of the locking member 20b may pass through the opening 38b in the proximal end portion 16b. As the tab 34b passes through the opening 38b in the proximal end portion 16b, the engagement of the locking member 20b with the proximal end portion 16b is released, the stopper 14 is free to move within the chamber 28 of the syringe barrel 12, and the stored energy of the biasing member 18 is released and applied to the stopper 14 moving the stopper 14 in a distal direction within the chamber 28 and expelling the medicinal fluid 21 from the chamber 28 through the open distal end 22 of the syringe barrel 12.

When the stopper 14 is in the second position and the medicinal fluid 21 has been expelled from the chamber 28 of the syringe barrel 12, the locking member 20b is completely contained within the chamber 28 of the syringe barrel 12 between the stopper 14 and the proximal end portion 16b.

The tab 34b may be shaped to provide gripping surfaces for a user to grasp the tab and rotate the tab 34b with respect to the proximal end portion 16b, for example, the large side surfaces of the disk shape of the tab 34b.

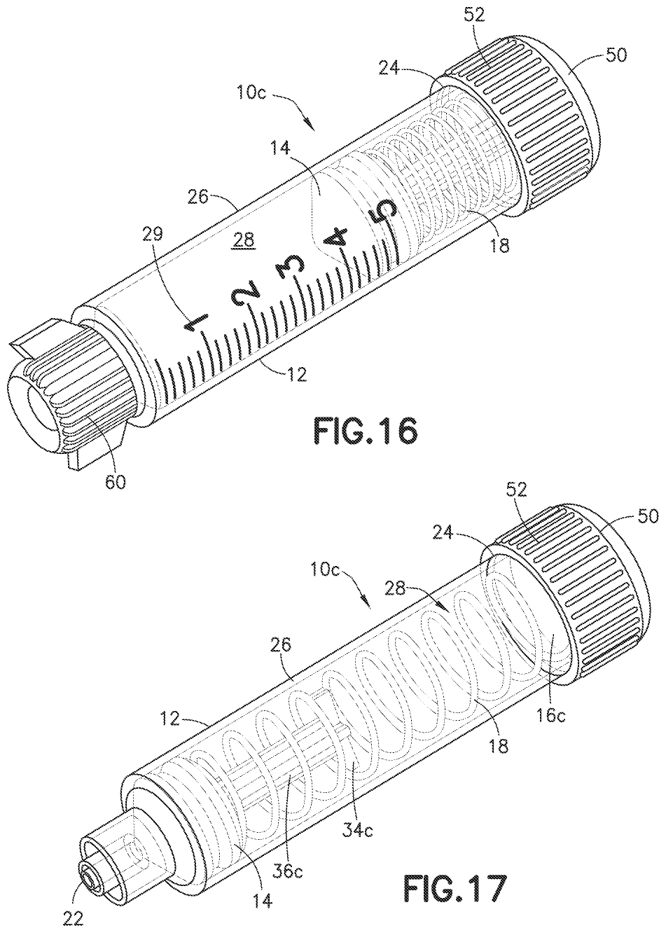

In a syringe having a third embodiment of the locking member 20c, shown in FIGS. 16-21, as with the second embodiment, when the stopper 14 is in the first position, the connection rod 36c extends through an opening 38c in the proximal end portion 16c and the tab 34c engages the proximal exterior surface 32c of the proximal end portion 16c. With the tab 34c engaging the proximal exterior surface 32c of the proximal end portion 16c, the stopper 14 is held in the first position and cannot move within the chamber 28 of the syringe barrel 12 (FIG. 16).

At least a portion of the tab 34c has a shape corresponding to the shape of at least a portion of the opening 38c, and the locking member 20c is transitionable from a locked position, where the tab 34c of the locking member 20c engages the proximal exterior surface 32c of the proximal end portion 16c, to a release position, where the tab 34c of the locking member 20c passes through the opening 38c in the proximal end portion 16c. The portion of the opening 38c may be a slit 46 and the portion of the tab 34c may be a bar 48. The opening 38c may further include a circular portion 49 through which the connection rod 36c extends.

The bar 48 may be perpendicular to the connection rod 36c such that the bar and the connection rod 36c form a T-shape (FIG. 21).

As with the second embodiment of the locking member 20b, in this embodiment of the locking member, with the tab 34c positioned perpendicular to the slit 46 (FIG. 20), the tab 34c engages the proximal exterior surface 32c of the proximal end portion 16c, the stopper 14 is held in the first position and cannot move within the chamber 28 of the syringe barrel 12 (FIG. 16). With the tab 34c positioned parallel to the slit 46, the tab 34c can pass through the opening 38c and into the chamber 28 of the syringe barrel 12. By rotating the locking member 20c with respect to the proximal end portion 16c, the locking member 20c can be transitioned from the locked position, where the tab 34c of the locking member 20c engages the proximal exterior surface 32c of the proximal end portion 16c, to the release position, where the tab 34c of the locking member 20c may pass through the opening 38c in the proximal end portion 16c. As the tab 34c passes through the opening 38c in the proximal end portion 16c, the engagement of the locking member 20c with the proximal end portion 16c is released, the stopper 14 is free to move within the chamber 28 of the syringe barrel 12, and the stored energy of the biasing member 18 is released and applied to the stopper 14 moving the stopper 14 in a distal direction within the chamber 28 and expelling the medicinal fluid 21 from the chamber 28 through the open distal end 22 of the syringe barrel 12.

When the stopper 14 is in the second position (FIG. 17) and the medicinal fluid 21 has been expelled from the chamber 28 of the syringe barrel 12, the tab 34c and the connection rod 36c are completely contained within the chamber 28 of the syringe barrel 12 between the stopper 14 and the proximal end portion 16c.

In this embodiment, the locking member 20c further comprises a locking member cap 50 covering the proximal end portion 16c. A portion 52 of the locking member cap 50 extends about an outer perimeter of the syringe barrel 12. The portion 52 of the locking member cap 50 extending about an outer perimeter of the syringe barrel 12 may include ridges to aid a user in gripping the locking member cap 50.

The locking member cap 50 releasably engages the tab 34c of the locking member 20c such that rotation of the locking member cap 50 with respect to the syringe barrel 12 rotates the locking member 20c with respect to the proximal end portion 16c, thereby transitioning the locking member 20c from the locked position to the release position and allowing the tab 34c to pass through the opening 38c, thereby releasing the engagement between the locking member 20c and the proximal end portion 16c.

Any suitable arrangement in which the locking member cap 50 engages the tab 34c of the locking member 20c to rotate the locking member 20c with respect to the proximal end portion 16c and releases the locking member 20c when the locking member 20c is in the release position may be provided. For example, protrusions 54 may extend from the bottom interior surface 56 of the locking member cap 50 and releasably engage the tab 34c of the locking member 20c. The protrusions 54 may define a recess 58 having a cross-section corresponding to a cross-section of the tab 34c, for example, the cross-sections of the recess 58 and the tab 34c may be cross-shaped, square, rectangular, triangular, or polygonal. The protrusions 54 act to rotate the locking member 20c while the engagement of the tab 34c in the recess 58 allows the tab 34c to pass through the opening 38c when the locking member 20c is in the release position.

Any of the additional features discussed below may be incorporated alone or in combination into any of the three embodiments discussed above.

Once the stopper 14 has reached the second position and the connection rod 36 or the entire locking member 20a, 20b, 20c is sealed within the chamber 28 of the syringe barrel 12 between the stopper 14 and the proximal end portion 16a, 16b, 16c, it is not possible to reuse the syringe 10a, 10b, 10c.

The biasing force provided to the stopper 14 by the biasing member 18 is greater than an opposing force asserted on the stopper 14. The opposing force is the sum of the frictional force between the stopper 14 and the sidewall 26 of the syringe barrel 12, the venous pressure coming from the attachment of the syringe 10a, 10b, 10c to the intravenous catheter, and the fluid pressure of the medicinal fluid 21 contained in the chamber 28 of the syringe barrel 12.

The materials of the stopper 14 and the sidewall 26 of the syringe barrel 12 may be chosen to provide a predetermined frictional force between the stopper 14 and the sidewall 26 of the syringe barrel 12. By setting the frictional force to a predetermined value, the amount by which the biasing force exceeds the opposing force can be adjusted. By adjusting the biasing force and the frictional force, the rate of movement of the stopper from the first position to the second position can be varied. For example, for a given biasing force, a higher frictional force will reduce the rate of movement of the stopper from the first position to the second position, and for a given frictional force, a lower biasing force will reduce the rate of movement of the stopper from the first position to the second position. Likewise, for a given biasing force, a lower frictional force will increase the rate of movement of the stopper from the first position to the second position, and for a given frictional force, a higher biasing force will increase the rate of movement of the stopper from the first position to the second position.

In addition, the biasing member 18 is configured to provide a predetermined amount of biasing force that, in combination with the diameter of the syringe barrel, will cause the medicinal fluid 21 to be expelled at a predetermined force. For example, for a biasing force of 22.25 N (5 lbs.) and a syringe barrel 12 having an inner diameter of 8.66 mm, the standard inner diameter for a 3 ml syringe, the medicinal fluid 21 will be expelled from the open distal end 22 of the syringe barrel 12 at a force of 379 kPa (55 psi), and for a biasing force of 22.25 N (5 lbs.) and a syringe barrel 12 having an inner diameter of 14.5 mm, the standard inner diameter for a 10 ml syringe, the medicinal fluid 21 will be expelled from the open distal end 22 of the syringe barrel 12 at a force of 136 kPa (19.75 psi). Preferably, the syringe barrel 12 has an inner diameter of 14 mm or greater and/or the biasing force applied by the biasing member 18 is 22.25 N (5 lbs.) or less, thereby reducing the pressure at which the medicinal fluid 21 is expelled from the open distal end 22 of the syringe barrel 12 and avoiding damage to the catheter.

The stopper 14 moves in a continuous manner from the first position to the second position expelling the medicinal fluid 21 from the open distal end 22 of the syringe barrel 12 in a continuous flow.

The biasing member 18 may be configured to only provide enough biasing force to the stopper 14 to move the stopper to the distal end of the chamber 28 of the syringe barrel 12 without providing enough biasing force to compress the stopper 14 against the distal bottom end of the syringe barrel 12, thereby maintaining positive pressure in the chamber 28 of the syringe barrel 12 and avoiding the creation of a negative pressure, which can cause reflux during disconnection of the syringe 10a, 10b, 10c from the catheter, if and when the stopper decompresses. For example, if the biasing member 18 is a spring, the length of the spring can be chosen to have a fully extended length that is insufficient to force the stopper 14 into the distal bottom end of the chamber 28 of the syringe barrel 12 when the stopper 14 is in the second position.

The syringe 10a, 10b, 10c may further include a removable tip cap 60 covering the open distal end 22 of the syringe barrel 12 prior to use.

The medicinal fluid 21 may be saline or an anticoagulant solution.

In use, the tip cap 60 is removed from the open distal end 22 of the syringe barrel 12 and the open distal end 22 of the syringe barrel 12 is connected to an intravenous catheter. The engagement between the locking member 20a, 20b, 20c and the proximal end portion 16a, 16b, 16c is then released, and the stopper 14 is automatically moved from the first position to the second position by the biasing member 18, thereby expelling the medicinal fluid 21 from the open distal end 22 of the syringe barrel 12 to flush the catheter. As described above, the locking member 20a may be released from engagement with the proximal end portion 16a by breaking the breakable connection 40 between the connection rod 36a and the tab 34a, the locking member 20b may be released from engagement with the proximal end portion 16b by using the tab 34b to rotate the locking member 20b with respect to the proximal end portion 16b, or the locking member 20c may be released from engagement with the proximal end portion 16c by rotating the locking member cap 50 with respect to the syringe barrel 12. After the flushing is completed, the syringe 10a, 10b, 10c may be disposed of in a suitable manner, for example, by being placed in a medical waste container.

The inventive syringe provides several advantages over manual prior art syringes in which the movement of the stopper from the first position to the second position is effected by the user pressing a plunger rod attached to the stopper.

With the inventive syringe, once the stopper 14 has reached the second position and the connection rod 36 or the entire locking member 20a, 20b, 20c is sealed within the chamber 28 of the syringe barrel 12 between the stopper 14 and the proximal end portion 16a, 16b, 16c, it is not possible to reuse the syringe 10a, 10b, 10c. Further, there is nothing provided to stop the automatic movement of the stopper 14, so a single syringe 10a, 10b, 10c cannot be used to provide flushing for catheters of different patients.

Also, the inventive syringe provides continuous flushing, where the medicinal fluid is continuously injected into the catheter. Continuous flushing has been found to be advantageous in reducing the amount of medicinal fluid required.

In addition, the inventive syringe 10a, 10b, 10c reduces the work required to flush a catheter by eliminating any need to manually prepare and fill a syringe with the medicinal fluid.

The inventive syringe also has a reduced packaging size as compared to prior art syringes because there is no plunger rod extending from the syringe barrel.

Whereas particular aspects of this invention have been described above for purposes of illustration, it will be evident to those skilled in the art that numerous variations of the details of the present invention may be made without departing from the invention.

It is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

1. A syringe comprising:
a syringe barrel comprising an open distal end, a proximal end, and a sidewall extending between the open distal end and the proximal end and defining a chamber;
a stopper disposed within the chamber in sealing engagement with the sidewall; and
wherein the syringe is configured to hold a medicinal fluid in the chamber of the syringe barrel between the stopper and the open distal end of the chamber,
characterized in that:
the syringe further comprises a biasing member configured to move the stopper from a first position to a second position;
a locking assembly comprising:
a proximal end member covering the proximal end of the syringe; and
a locking member connected to the stopper,
wherein at least a portion of the locking member engages the proximal end member to hold the stopper in the first position, disengagement of the portion of the locking member from the proximal end member frees the stopper, and the stopper is moved to the second position by the biasing member.

2. The syringe of claim 1, wherein the locking member comprises a tab and a connection rod extending between the tab and the stopper.

3. The syringe of claim 2, wherein, when the locking member is engaged with the proximal end portion, the connection rod extends through an opening in the proximal end portion and the tab engages a proximal exterior surface of the proximal end portion.

4. The syringe of claim 3, wherein the tab is sized and/or shaped such that the tab cannot pass through the opening.

5. The syringe of claim 2, wherein the tab is releasably attached to the connection rod and detachment of the tab from the connection rod disengages the locking member from the proximal end portion.

6. The syringe of claim 5, wherein the tab is attached to the connection rod by a breakable connection by which the tab can be detached from the connection rod.

7. The syringe of claim 3, wherein at least a portion of the tab has a shape corresponding to a shape of at least a portion of the opening, and the locking member is transitionable from a locked position, where the tab of the locking member engages the proximal end portion to a release position where the tab of the locking member passes through the opening in the proximal end portion.

8. The syringe of claim 7, wherein rotation of the locking member with respect to the proximal end portion transitions the locking member from the locked position to the released position.

9. The syringe of claim 7, wherein the proximal end portion is contained within the chamber at the proximal end of the syringe barrel and the locking member further comprises a locking member cap covering the proximal end portion, the locking member cap engaging the tab.

10. The syringe of any of claim 9, wherein rotation of the locking member cap with respect to the syringe barrel transitions the locking member from the locked position to the release position.

11. The syringe of claim 9, wherein protrusions extend from the locking member cap, define a recess having a cross-section corresponding to a cross-section of the tab, and engage the tab.

12. The syringe of claim 9, wherein the portion of the opening is a slit and the portion of the tab is a bar.

13. The syringe of claim 1, wherein the stopper moves in a continuous manner from the first position to the second position expelling the fluid from the syringe in a continuous flow.

14. The syringe of claim 1, wherein the biasing member comprises a compression spring.

15. The syringe of claim 1, wherein the biasing member comprises a viscous damper.
